(19) United States
(12) Patent Application Publication
(10) Pub. No.: US 2023/0364272 A1
(43) Pub. Date: Nov. 16, 2023

(54) THERANOSTIC COMPOUNDS

(71) Applicants: NRF ITHEMBA LABS, Cape Town (ZA); UNIVERSITY OF CAPE TOWN, Cape Town (ZA); THE SOUTH AFRICAN NUCLEAR ENERGY CORPORATION LIMITED, Brits Magisterial District (ZA)

(72) Inventors: Charlot Rosa VANDEVOORDE, Cape Town (ZA); Daniel Du Toit ROSSOUW, Cape Town (ZA); Denver Thomas HENDRICKS, Cape Town (ZA); Jacobus Petrus SLABBERT, Cape Town (ZA); Jan Rijn ZEEVAART, Brits Magisterial District (ZA); Julie Anne BOLCAEN, Cape Town (ZA); Roger HUNTER, Cape Town (ZA); Shankari NAIR, Cape Town (ZA); Virna Drucille LEANER, Cape Town (ZA)

(21) Appl. No.: 18/041,313

(22) PCT Filed: Aug. 13, 2021
(86) PCT No.: PCT/IB2021/057465

§ 371 Date: Feb. 10, 2023

(30) Foreign Application Priority Data
Aug. 13, 2020 (GB) 2012671.0

(51) Int. Cl. A61K 51/04; C07B 59/00; A61P 35/00

(52) U.S. Cl. CPC A61K 51/0402; C07B 59/001; A61P 35/00; A61B 6/037

(57) ABSTRACT

This invention relates to a hydroxamate metalloprotease inhibitor compound for use in a method of diagnosing or treating cancer, inflammatory diseases or Alzheimer's disease. The compound comprises a zinc-chelating N-hydroxamate moiety radiolabeled with a radionuclide. Radiolabeled compounds of the invention may be used in targeted radionuclide therapy wherein a patient is treated with a compound of the invention comprising a diagnostic radionuclide to identify the presence of a cancer or disease, followed by treatment with a compound of the invention comprising a therapeutic radionuclide to treat said cancer or disease.

### BACKGROUND TO THE INVENTION

Globally, cancer affects 14 million people and accounts for one fifth of deaths in both developing and developed countries. The World Health Organization (WHO) reports that one out of three people will be diagnosed and treated for some form of life-threatening cancer in their life time, making cancer one of the leading causes of death after heart disease. Incidences of most cancers increase with age and these numbers continue to rise with increasing life expectancy. In sub-Saharan Africa, prostate and lung cancer is most prevalent in males; breast and cervical cancer are the most common types to affect women. Cervical and esophageal cancers are among the most common cancers affecting Southern Africans and do not generally present symptoms until a late stage. Hence, diagnosis is likely only made after the cancer has spread or is at an advanced stage. According to reports from CANSA (Cancer Association of South Africa), cervical cancer kills more women in Southern Africa than any other form of cancer, except for breast cancer. As such, it is important to focus on the detection and treatment of these high incidence cancers and their impact in a Southern African setting. Conventionally, there are four approaches to cancer treatment after diagnosis: surgery, chemotherapy, radiotherapy and palliative care. Radiotherapy or radiation therapy (RT) can be an effective treatment, especially for localized or solid cancers and about half of cancer patients receive radiation as a curative or palliative treatment. Radiotherapy uses high energy radiation to control cancer cell growth by damaging DNA within the cancer cell. If the DNA of a cancer cell is sufficiently damaged, the cells are unable to replicate as usual and the growth of the tumor is inhibited. Different types of irradiation, such as X-rays, gamma rays, electron beams, protons, and/or charged particles are irradiated at the tumor site either from outside (external-beam radiotherapy) or inside (internal radiotherapy) the body. However, although RT alone or combined with other modalities is effective for treating cancer globally, some cancers are not responding. This could be due to the development of radiation resistance but it depends also on the cancer type, location and an early diagnosis is important. Therefore, attempts to combine RT with cellular and molecular targeted biological modalities which determine the sensitivity or resistance to ionizing radiation is an unmet need. Secondly, new targeted therapies are urgently needed that only kill cancer cells without inducing normal tissue toxicity. Thirdly, new imaging probes with a higher specificity and appropriate for early cancer detection are needed as early detection is often the key to surviving any form of cancer. These objectives can be achieved through the radiolabeling of small molecule inhibitors, which can be used for the detection as well as the treatment of specific tumor types.

It is an object of the present invention to provide a new radiolabeled pharmaceutical compound for the early detection and treatment of cancer, in particular cervical and esophageal cancer.

### SUMMARY OF THE INVENTION

According to the present invention there is provided a hydroxamate metalloprotease inhibitor compound for use in a method of diagnosing or treating cancer, inflammatory diseases or Alzheimer's disease, comprising a zinc-chelating N-hydroxamate moiety radiolabeled with a radionuclide, the zinc-chelating N-hydroxamine moiety, having the general structure:

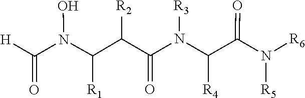

wherein:

R₁ is an alkyl group, preferably a short alkyl chain such as methyl or ethyl;

R₂ is an alkyl, alkenylene, alkynylene, or aryl group, such as 3-phenyl-1-propyl, alkenylene, or alkynylene;

R₃ is H or lower alkyl;

R₄ is an alkyl, alkenylene, alkynylene, or aryl group, such as tert-butyl; alkenylene, alkynylene, or arylene;

R₅ is H or lower alkyl; and

R₆ is an alkyl group, preferably a short alkyl chain such as methyl.

The radionuclide is preferably incorporated into an aryl group such as a phenyl ring on the R₂ moiety, typically in the para-position.

The hydroxamate metalloprotease inhibitor is typically G1254023X ((2R,3S)-3-(Formylhydroyamino)-2-(3-phenyl-1-propyl)butanoic Acid [(1S)-2,2-Dimethyl-1-(methylcarbamoyl)-1-propyl]amide) and derivatives thereof.

In the case where the compound is for use in a method of diagnosing cancer, inflammatory diseases or Alzheimer's disease, the radionuclide may be a diagnostic radionuclide selected from ^{99m}Tc, ¹⁸⁸Re, ¹⁸⁶Re, ¹⁵³Sm, ⁶⁷Ga, ⁶⁸Ga, ¹¹¹In, ⁵⁹Fe, ⁶³Zn, ⁵²Fe, ⁴⁵Ti, ⁶⁰Cu, ⁶¹Cu, ⁶⁷Cu, ⁶⁴Cu, ⁶²Cu, ¹⁹⁸Au, ¹⁹⁹Au, ^{195m}Pt, ^{191m}Pt, ^{193m}Pt, ^{117m}Sn, ⁸⁹Zr, ¹⁷⁷Lu, ¹⁸F, ⁷⁴Br, ⁷⁵Br, ⁷⁶Br, ⁷⁷Br, ⁸⁰Br, ⁸²Br, ⁸⁵Br and ¹²³I.

In the case where the compound is for use in a method of treating cancer, inflammatory diseases or Alzheimer's disease, the radionuclide may be a therapeutic radionuclide selected from ¹⁸⁸Re, ¹⁸⁶Re, ¹⁵³Sm, ¹⁶⁶Ho, ⁹⁰Y, ⁸⁹Sr, ¹¹¹In, ¹⁵³Gd, ²²⁵Ac, ²¹²Bi, ²¹³Bi, ²¹¹At, ⁶⁰Cu, ⁶¹Cu, ⁶⁷Cu, ⁶⁴Cu, ⁶²Cu, ¹⁹⁸Au, ¹⁹⁹Au, ^{195m}Pt, ^{193m}Pt, ¹⁹⁷Pt, ^{117m}Sn, ¹⁰³Pd, ^{103m}Rh, ¹⁷⁷Lu, ²²³Ra, ²²⁴Ra, ²²⁷Th, ³²P, ¹⁶¹Tb, ³³P, ¹²⁴I, ¹²⁵I, ¹³¹I, ²⁰³Pb, ²⁰¹Tl, ¹¹⁹Sb, ^{58m}Co, ⁷⁴Br, ⁷⁵Br, ⁷⁶Br, ⁷⁷Br, ⁸⁰Br, ⁸²Br, ⁸⁵Br and ¹⁶¹Ho.

Radiolabeled compounds of the invention may be used in targeted radionuclide therapy wherein a patient is treated with a compound of the invention comprising a diagnostic radionuclide to identify the presence of a cancer or disease, followed by treatment with a compound of the invention comprising a therapeutic radionuclide to treat said cancer or disease. Typical radionuclides are isotopes of I and Br. For example, this therapy could be achieved by injecting a patient with a compound of the invention radiolabeled with diagnostic radionuclide ¹²³I and identifying a tumor or disease using SPECT imaging or ¹²⁴I for PET imaging, and then exchanging the radionuclide with a therapeutic radionuclide such as ¹²⁵I or ¹³¹I and injecting said patient for therapy.

The cancer may be cervical or esophageal cancer.

In another embodiment of the invention, the cancer is glioblastoma.

The invention also covers methods of medical treatment using the compounds and therapies described above.

The invention further covers methods of producing a radiolabeled hydroxamate metalloprotease inhibitor described above.

The radiolabeled compound may be synthesized by the direct radio-iodination on the aromatic ring of said compound or a precursor using S_{E}Ar aromatic substitution chemistry methodology with N-chlorosuccinimide in trifluoroacetic acid.

### BRIEF DESCRIPTION OF THE DRAWINGS

FIG. 1 is UV chromatographs of the compounds ¹²³I-SN-311, I-SN-311 and SN-311;

FIG. 2 is a graph showing the Uptake of [¹²³I]Nal and ¹²³I-SN-311 in HeLa and WHCO5 cells as a function of Bq activity per well as a function of cell number; and

FIG. 3 is a graph showing Total Uptake in WHCO5 and HeLa cells of ¹²³I-SN-311.

### DESCRIPTION OF PREFERRED EMBODIMENTS

The aim of the invention is to synthesize a new theranostic radiopharmaceutical. The theranostic approach in nuclear medicine couples diagnostic imaging and therapy using the same molecule or at least very similar molecules, which are either radiolabeled with a different isotope or administered in different dosages. A theranostic agent needs to have two functions:
1. Diagnostic: to find a specific molecular target which is present and (over)expressed in a certain type of cancer, that can be quantified and located.
2. Therapeutic: delivering a targeted radiation treatment to the same molecular target (over)expressed by the cancer cells.

The premise for this theranostic approach is that you only treat what you can visualize and that the efficacy of therapy can be quantified and monitored. Theranostic radiopharmaceuticals are therefore a useful tool in the development of personalized medicine approaches where the therapy is tailor made for the individual patient. In this context, it is very important to select a drug target that is only present or overexpressed on a tumor cell, such as a specific receptor, compared to healthy cells throughout the body. Secondly, a drug needs to be synthesized that binds to this specific cancer target. If this is the case, radiolabeling the drug with an imaging or a therapeutic radioisotope could enable to image the tumor or deliver a radiation dose that can kill the tumor cells, respectively.

A preferred compound is the compound G1254023X ((2R,3S)-3-(Formylhydroyamino)-2-(3-phenyl-1-propyl)butanoic Acid [(1S)-2,2-Dimethyl-1-(methylcarbamoyl)-1-propyl]amide) from GlaxoSmith Kline (GSK) described in WO2000/0012083, the content of which is incorporated herein by reference.

G1254023X has three stereogenic centers, as well as the characteristic N-formyl hydroxylamine functionality. The latter can occur in either E- or Z-configurations which, together with the three centers of chirality, make four stereogenic elements that result in eight possible diastereomers of the inhibitor as racemates (sixteen stereoisomers in total).

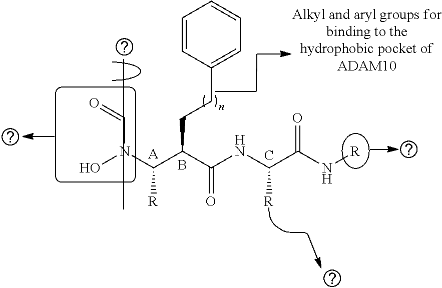

The alkyl substituents are preferred at position A (hereon referred to as carbon A), while the substituent at position B (hereon referred to as carbon B) tolerates both alkyl and aryl groups for binding in the hydrophobic pocket of the enzyme. In addition, S-stereogenicity orientation is preferred at position C, ensuring the substituent faces away from the binding pocket of the enzyme and points towards the solvent. Finally, a short alkyl chain, such as a methyl group, is favored as the N-alkyl substituent of the secondary amide at the C-terminus.

According to the present invention, the radionuclide is incorporated into the phenyl ring of the side chain at stereo-center B.

The structure below shows modified G1254023X labelled with the radionuclide ¹²³I.

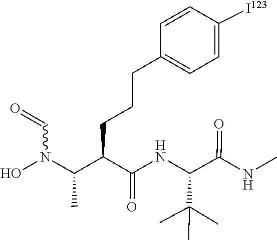

Radiolabeling Approaches/Methodologies Included in this Invention

### 1. SN-311+¹²³I (Diagnostic Isotope)

In one embodiment of the invention, the radiolabeled compound is achieved by the direct radio-iodination on the aromatic ring of the precursor, which is referred to as SN-311. The synthesized compound G1254023X (syn-) differs only in its relative stereochemistry to G1254023X (anti-). Primarily studies indicated very little difference between the two for the ADAM10 receptor uptake, with in vitro tests on the radiolabeled compound confirming cell binding affinity as expected. Synthetic challenges were encountered during the synthesis, which primarily emanated from a failure to reproduce the stereoselective alkylation of methyl-3-hydroxybutyrate with cinnamyl bromide in the original G1254023X synthesis (WO2000012083). This resulted in the adoption of a strategy to use an Evans syn-aldol reaction to accomplish the synthesis of C-2/C-3 ‘syn’ variants (see the Figure above) of G1254023X: SN-254 and SN-311. However, only SN-311 was taken into the radiolabeling studies, due to its improved biological activity. ADAM10 biological activity was measured for SN-311 and EC₅₀ values in cervical (HeLa) and esophageal (WHCO5) cancer cell lines were found to fall within the 95% confidence interval of that of G1254023X. This indicated that the C-2/C-3 relative stereochemistry in G1254023X is not biologically determining and that SN-311 could be radiolabeled with ¹²³I.

### 1.1 Synthesis of Compounds SN-254 and SN-311

### SN-254 Synthesis

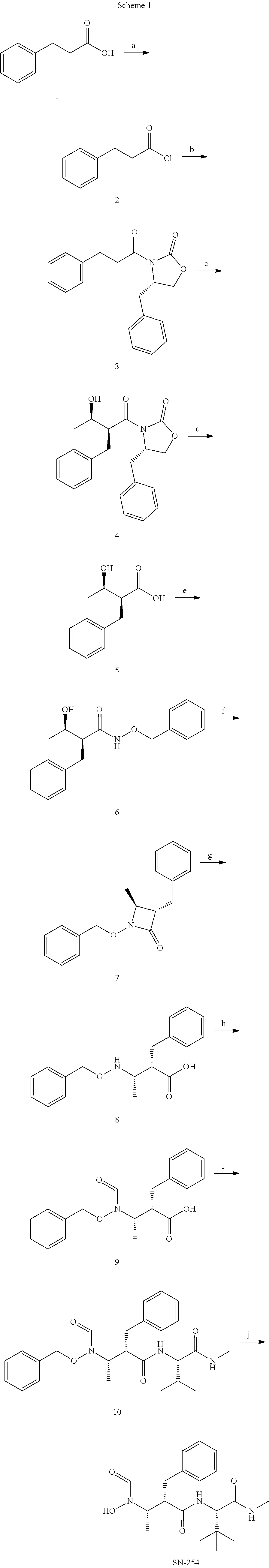

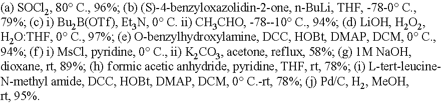

(S)-4-Benzyloxazolidin-2-one was coupled to 2 to give the N-acyl oxazolidinone derivative, 3. The boron-mediated stereoselective aldol reaction of 3 produced 4 with C-2/C-3 syn-relative stereochemistry and (2S, 3R)-absolute stereochemistry. The oxazolidinone auxiliary was removed and the resultant acid coupled to O-benzylhydroxylamine to generate 6. Following this, 6 was subjected to a two-step sequence involving mesylation followed by base-mediated intramolecular cyclisation to generate the β-lactam, 7. Thereafter, the lactam ring was hydrolyzed to produce acid, 8, which was N-formylated with formic acetic anhydride to synthesize 9. Afterwards, 9 was reacted with L-tert-leucine N-methyl amide to produce amide, 10. Finally, hydrogenolysis of 10 produced SN-254 in 10 linear steps, with (2S,3S)-stereochemistry.

### SN-311 Synthesis

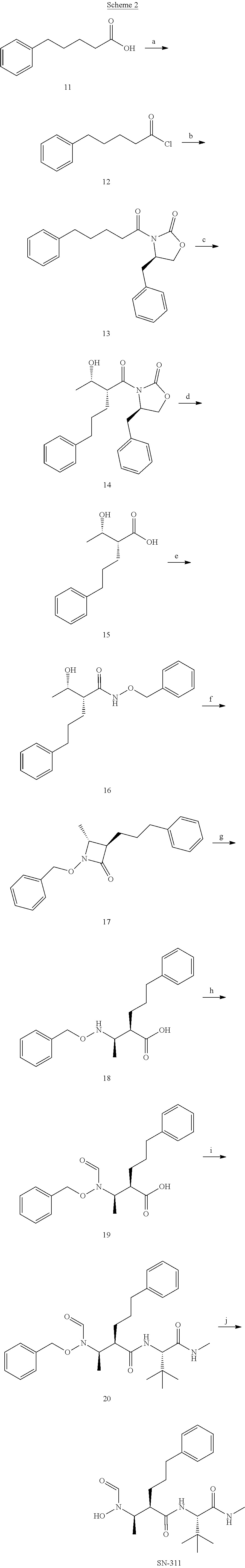

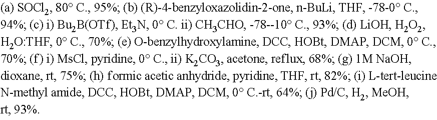

(R)-4-Benzyloxazolidin-2-one was coupled to 12 to give the N-acyl oxazolidinone derivative, 13, which underwent a boron-mediated stereoselective aldol reaction to produce 14. The oxazolidinone auxiliary was removed and the resultant acid coupled with O-benzylhydroxylamine to produce 16. Thereafter, 16 was subjected to mesylation and base-mediated intramolecular cyclisation to generate the β-lactam, 17. Subsequently lactam ring was hydrolyzed to produce acid, 17, which underwent N-formylation reaction using formic acetic anhydride to afford, 19. Then, 19 was coupled with L-tert-leucine N-methyl amide to yield amide, 20, with subsequent hydrogenolysis producing SN-311 in 10 linear steps, with (2R,3R)-stereochemistry.

### 1.2 Iodination of SN-311 (I-SN-311)

### Synthesis

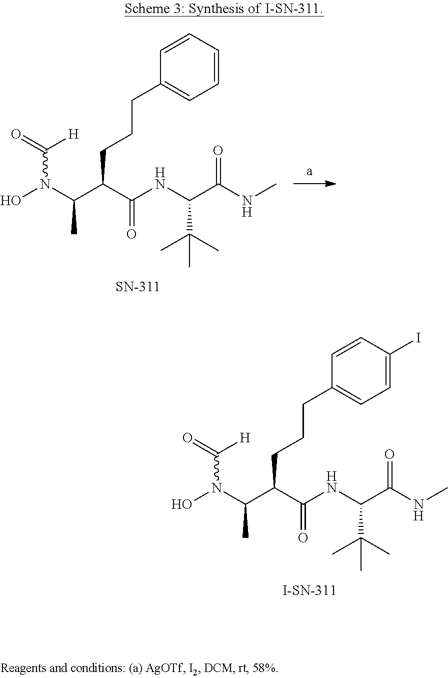 [SMILES: CNC(=O)[C@@H](NC(=O)[C@H](CCCc1ccccc1)[C@@H](C)N(O)C=O)C(C)(C)C ; CNC(=O)[C@@H](NC(=O)[C@H](CCCc1ccc(I)cc1)[C@@H](C)N(O)C=O)C(C)(C)C ; "I-SN-311"]

I-SN-311 was produced by reacting iodine with SN-311 and silver triflate in a S_{E}Ar reaction (via I⁺). The product was isolated as a brown oil in 58% yield.

### 1.3 Radioiodination of SN-311 (¹²³I-SN-311)

### Synthesis

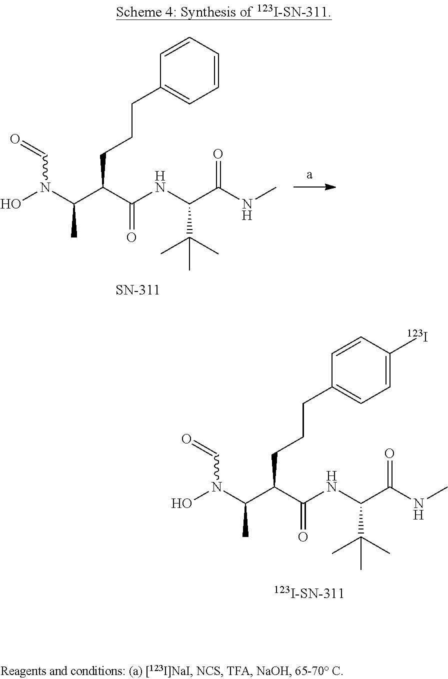 [SMILES: CNC(=O)[C@@H](NC(=O)[C@H](CCCc1ccccc1)[C@@H](C)N(O)C=O)C(C)(C)C ; CNC(=O)[C@@H](NC(=O)[C@H](CCCc1ccc([123I])cc1)[C@@H](C)N(O)C=O)C(C)(C)C ; "123I-SN-311"]

The radiolabeling was also achieved using an S_{E}Ar reaction by adding [¹²³I]Nal in dilute NaOH solution to a solution of SN-311 and N-chlorosuccinimide in trifluoroacetic acid, with heating of the mixture for approximately 2.5 hours at 65-70° C. The radiolabeled material was isolated on a reversed phase C18 Sep-Pak mini-cartridge column after preparation of the column using 5 mL of de-ionized water followed by 6 mL ethanol.

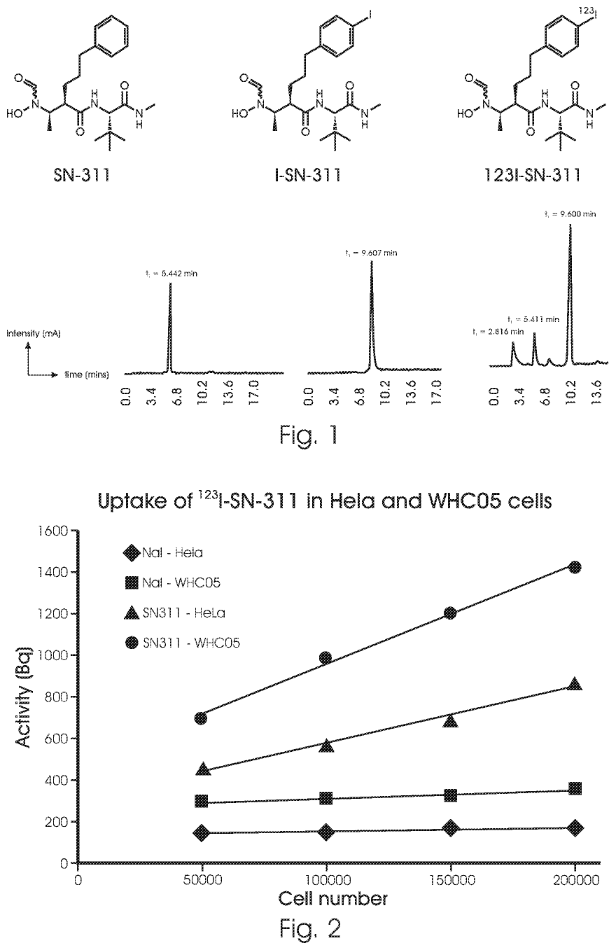

The HPLC chromatographs (UV detector) are shown in FIG. 1, where it was determined that ¹²³I was incorporated on the aromatic ring of compound SN-311, by virtue of a peak with the same retention time in both the ‘cold’ and radiolabeled samples at 9.6 minutes. From the retention times of traces B and C, it could be concluded that substitution in the radiolabeled run was predominantly para- as in the silver triflate/iodine method (verified by ¹H NMR spectroscopy). The radiolabeled material was then immediately added to a solution of 0.15 M phosphate-buffered saline at pH 7.4, which was done to safely transport the radioactive material to the cell cultures for evaluation in cancer cell lines.

### 2. Radioiodination of GI254023X

### Synthesis

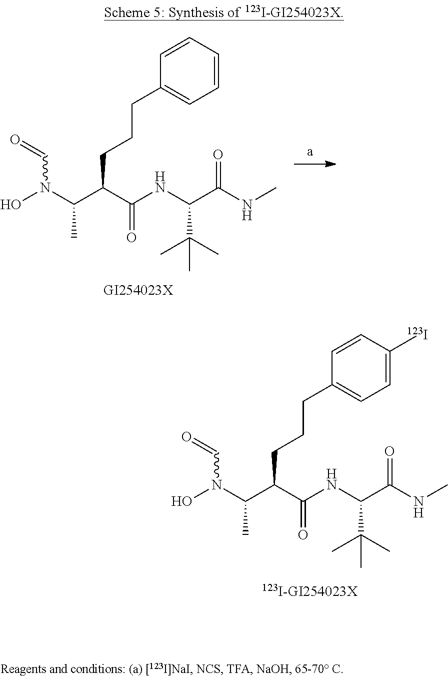 [SMILES: CNC(=O)[C@@H](NC(=O)[C@H](CCCc1ccccc1)[C@H](C)N(O)C=O)C(C)(C)C ; CNC(=O)[C@@H](NC(=O)[C@H](CCCc1ccc([123I])cc1)[C@H](C)N(O)C=O)C(C)(C)C ; "123I-GI254023X"]

¹²³I-G1254023X was synthesized using [¹²³I]Nal in dilute NaOH with N-chlorosuccinimide in trifluoroacetic acid

### 3. GI254023X+Theranostic Isotope

As mentioned above, the goal is not only to develop an imaging agent but a theranostic agent, which includes also a therapeutic radioisotope. The idea is that when ¹²³I-G1254023X SPECT imaging is able to identify the presence of ADAM10 in the tumor, the patient would be a candidate for targeted ADAM10 radionuclide therapy. This therapy could be achieved by subsequently injecting radiolabeled GI254023X but now exchanging the diagnostic radionuclide (¹²³I) with a therapeutic one: ¹²⁵I (therapy) or ¹³¹I (therapy). A further aspect of the invention is the use of this radiolabeled theranostic ADAM10 inhibitor for other clinical applications, such as other types of cancers (next to glioblastoma, cervical and esophageal cancer that are described in the pre-clinical investigation below) and perhaps even Alzheimer's disease ¹²³I-G1254023X and ¹³¹I-G1254023X could be a very interesting theranostic application, particularly for glioblastoma. In the following sections, the first potential clinical application of the invention will be described in more detail with reference to preliminary results of pre-clinical investigations.

ADAM10 biological activity was conducted on SN-311 with EC₅₀ values in cervical (HeLa) and esophageal (WHCO5) cancer cell lines found to fall within the 95% confidence interval of that of G1254023X. This indicated that the C-2/C-3-relative stereochemistry in G1254023X is not biologically determining and that SN-311 could be radiolabeled with ¹²³I. Radioactively-labelled compound ¹²³I-SN311 with the γ-emitter ¹²³I was synthesized as a potential radiodiagnostic, following the direct iodination of compound SN-311 with [¹²³I]Nal. ¹²³I-SN-311 was successfully synthesized using [¹²³I]Nal in dilute NaOH solution with N-chlorosuccinimide in trifluoroacetic acid using S_{E}Ar aromatic substitution chemistry methodology. The substitution regioselectivity was para- on the aromatic ring, with the rest of the molecule left intact as determined by HPLC comparison with a synthesized ‘cold’ equivalent, the latter carefully evaluated by ¹H and ¹³C NMR spectroscopies.

Cellular uptake studies demonstrated that the radiolabeled compound (¹²³I-SN-311) was taken up into cervical cancer cell lines. ¹²³I-SN-311 was proposed to be used as a radiopharmaceutical for the early diagnosis of cervical cancer. A further possibility is to exchange the diagnostic radionuclide with a therapeutic isotope. (Iodine: I-123 for SPECT, I-124 for PET; I-131 and I-125 for therapy).

¹²³I-G1254023X was synthesized using [¹²³I]Nal in dilute NaOH with N-chlorosuccinimide in trifluoroacetic acid using S_{E}Ar aromatic substitution chemistry methodology. All in vitro radiobiology studies thus far have been carried out using ¹²³I-G1254023X.

As mentioned, not only detection is possible, but also treatment by exchanging the diagnostic radionuclide with a therapeutic one using the following pairs. I-123 for SPECT, I-124 for PET, I-131 and I-125 for therapy. Isotopes of Br can also be used.

A further aspect of the invention is the use of the radiolabeled inhibitor, ¹²³I-G1254023X in other applications in theranostic. The proposed new application will use ¹²³I-G1254023X as a radiopharmaceutical in other types of cancers (only cervical cancer is being investigated in concepts 1 and 2 above). ¹²³I-G1254023X and potentially ¹³¹I-G1254023X can be used in the diagnosis and treatment of glioblastoma.

### First Pre-Clinical Results on the Radiolabeled Compound Cervical Cancer

In order to test the biological activity of the radiolabeled compound in vitro, two cell lines were chosen as esophageal (WHCO5) and cervical (HeLa) cancer cell lines (using [¹²³I]Nal as the control) and the radioactivity uptake was measured using a bore-hole or scintillation counter. The method involved administration of the ¹²³I radiolabeled ADAM 10 inhibitor, which was diluted in complete cell growth medium and added to the cultures to give a final concentration of 3 μCi per well or 0.3 μCi per well depending on the assay. The well plates were incubated for one hour in a humidified CO₂ incubator at 37° C. The measurements were performed with a ¹²³I Multi Channel Analyzer, and during the incubation time, a calibration and counting efficiency determination was carried out on the channel.³³

After the one-hour incubation period, the growth medium was aspirated, leaving the adherent cell monolayers intact and the cell cultures rinsed twice with cold PBS to remove any residual extracellular radioactivity. The cell monolayers were lysed with 1 mL 1M NaOH, and 1 mL of each lysed suspension was transferred to a clean test tube. Radioactive counts and corrections applied for different cell numbers per well are listed in Table 1. The data is presented as an average of triplicates and was corrected for ¹²³I decay using Eff Corr as the correcting factor, which considers the half-life and decay of the radionuclide over a specific time interval.

TABLE 1
Uptake of [¹²³I]NaI and ¹²³I-46 (3 mCi) in HeLa and WHCO5 cancer cells

| Condition | [¹²³I]NaI - HeLa | | | | [¹²³I]NaI - WHCO5 | | | |
|---|---|---|---|---|---|---|---|---|
| Number of cells | 50000 | 100000 | 150000 | 200000 | 50000 | 100000 | 150000 | 200000 |
| Average counts per min | 1974 | 1991 | 2308 | 2147 | 3968 | 4116 | 4239 | 4772 |
| A(0) per well × Eff corr | 8857 | 8934 | 10355 | 9635 | 17807 | 18470 | 19021 | 21415 |
| Bq per well | 148 | 149 | 173 | 161 | 297 | 308 | 317 | 357 |
| Condition | ¹²³I-SN-311 HeLa | | | | ¹²³I-SN-311 WHCO5 | | | |
| Number of cells | 50000 | 100000 | 150000 | 200000 | 50000 | 100000 | 150000 | 200000 |
| Average counts per min | 6080 | 7582 | 9236 | 11555 | 9337 | 13207 | 16075 | 18996 |
| A(0) per well × Eff corr | 27284 | 34023 | 41445 | 51852 | 41898 | 59265 | 72135 | 85240 |
| Bq per well | 455 | 567 | 691 | 864 | 698 | 988 | 1202 | 1421 |

FIG. 2 presents the activity (Bq) per well as a function of cell number and is a graphical representation of data listed in Table 1. Results are the mean +/− SEM of experiments performed in triplicate. For both cell lines, no appreciable increase in the activity of [¹²³I]Nal with cell number was observed, whereas with the inhibitor (green and blue circles), there was a significant uptake, particularly in the WHCO cell line.

Free [¹²³I]Nal showed minimal uptake in both cells lines, although the base line readings were higher in WHCO5 cells as compared to HeLa cells (Table 1 and FIG. 8). There is a clear uptake of ¹²³I-SN-311 in both HeLa and WHCO5 cancer cell lines, with both showing a linear increase in uptake with an increase in the number of cells. The faster-growing WHCO5 cells show more uptake of radioactivity, and this corresponds to the literature, which reports that faster growing cell lines have an increased uptake of substances.³⁴

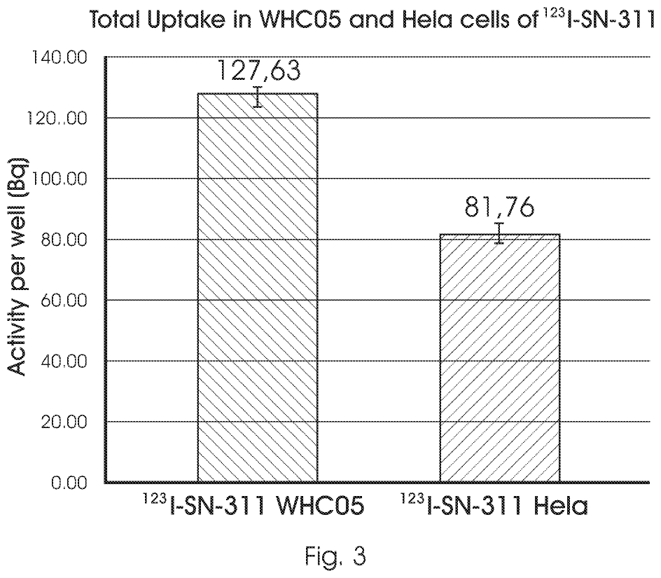

A second set of readings were made using only 0.3 μCi per well, the principle reason for this was to reduce the influence of non-specific binding to the plastic of each well. The data is summarized in FIG. 3. Data represents an average and standard deviation of 12 replicates using 0.3 μCi of ¹²³I-SN-311 and 200000 cells.

Again, the faster growing WHCO5 cells showed significantly higher uptake of the radiolabeled compound (127.63 Bq) compared to the slower growing HeLa cells (81.76 Bq).

The similar and comparable radiolabeling results of G1254023X and SN-311 further demonstrated that the stereochemical configurational attributes were not crucial for uptake. Importantly, the uptake studies have demonstrated that the radiolabeled compound (¹²³I-SN-311) was taken up into HeLa and WHCO5 cancer cells.

### Glioblastoma

The synthesized radiopharmaceutical has potential as theranostic agent for growth inhibition of glioblastoma (GB), as well as for GB tumor imaging. GB comprise high-grade gliomas (HGG), and is the most aggressive and most common malignant brain tumor in adults, with a high mortality and morbidity. ADAM10 promotes glioma migration and invasion and has been identified as a promising prognostic factor. Expression of ADAM10 was confirmed in 22-64% of GB specimens while it was absent in the normal brain. ADAM10 inhibition has been shown to boost an immune response against GB-initiating cells and to sensitize GB cells to therapy.

1. A hydroxamate metalloprotease inhibitor compound for use in a method of diagnosing or treating cancer, inflammatory diseases or Alzheimer's disease, comprising a zinc-chelating N-hydroxamate moiety radiolabeled with a radionuclide, the zinc-chelating N-hydroxamine moiety, having the general structure: 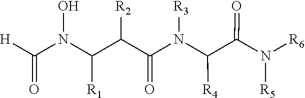
wherein:
R₁ is an alkyl group;
R₂ is an alkyl, alkenylene, alkynylene, or aryl group;
R₃ is H or lower alkyl;
R₄ is an alkyl, alkenylene, alkynylene, or aryl group;
R₅ is H or lower alkyl; and
R₆ is an alkyl group.

2. The compound claimed in claim 1, wherein R₁ is a short alkyl chain.

3. The compound claimed in claim 2, wherein R₁ is methyl or ethyl.

4. The compound claimed in claim 1, wherein R₂ is 3-phenyl -1-propyl; alkenylene, or alkynylene.

5. The compound claimed in claim 1, wherein R₄ is as tert-butyl; alkenylene, alkynylene, or arylene.

6. The compound claimed in claim 1, wherein R₆ is a short alkyl chain.

7. The compound claimed in claim 6, wherein R₆ is methyl.

8. The compound claimed in claim 1, wherein the radionuclide is incorporated into an aryl group on the R₂ moiety.

9. The compound claimed in claim 8, wherein the aryl group is a phenyl ring.

10. The compound claimed in claim 9, wherein the radionuclide is incorporated into the phenyl ring in the para-position.

11. The compound claimed in claim 1 which is G1254023X ((2R,3S)-3-(Formylhydroyamino)-2-(3-phenyl-1-propyl)butanoic Acid [(1S)-2,2-Dimethyl-1-(methylcarbamoyl)-1-propyl]amide) and derivatives thereof.

12. A method of using a compound as claimed in claim 1 comprising using the compound for diagnosing cancer, inflammatory diseases or Alzheimer's disease, wherein the radionuclide is a diagnostic radionuclide selected from ^{99m}Tc, ¹⁸⁸Re, ¹⁸⁶Re, ¹⁵³Sm, ⁶⁷Ga, ⁶⁸Ga, ¹¹¹In, ⁵⁹Fe, ⁶³Zn, ⁵²Fe, ⁴⁵Ti, ⁶⁰Cu, ⁶¹Cu, ⁶⁷Cu, ⁶⁴Cu, ⁶²Cu, ¹⁹⁸Au, ¹⁹⁹Au, ^{195m}Pt, ^{191m}Pt, ^{193m}Pt, ^{117m}Sn, ⁸⁹Zr, ¹⁷⁷Lu, ¹⁸F, ⁷⁴Br, ⁷⁵Br, ⁷⁶Br, ⁷⁷Br, ⁸⁰Br, ⁸²Br, ⁸⁵Br and ¹²³I.

13. A method of using a compound as claimed in claim 1 comprising using the compound for treating cancer, inflammatory diseases or Alzheimer's disease, wherein the radionuclide is a therapeutic radionuclide selected from ¹⁸⁸Re, ¹⁸⁶Re, ¹⁵³Sm, ¹⁶⁶Ho, ⁹⁰Y, ⁸⁹Sr, ¹¹¹In, ¹⁵³Gd, ²²⁵Ac, ²¹²Bi, ²¹³Bi, ²¹¹At, ⁶⁰Cu, ⁶¹Cu, ⁶⁷Cu, ⁶⁴Cu, ⁶²Cu, ¹⁹⁶Au, ¹⁹⁹Au, ^{195m}Pt, ^{193m}Pt, ¹⁹⁷Pt, ^{117m}Sn, ¹⁰³Pd, ^{103m}Rh, ¹⁷⁷Lu, ²²³Ra, ²²⁴Ra, ²²⁷Th, ³²P, ¹⁶¹Tb, ³³P, ¹²⁴I, ¹²⁵I, ¹³¹I, ²⁰³Pb, ²⁰¹Tl, ¹¹⁹Sb, ^{58m}Co, ⁷⁴Br, ⁷⁵Br, ⁷⁶Br, ⁷⁷Br, ⁸⁰Br, ⁸²Br, ⁸⁵Br and ¹⁶¹Ho.

14. The method of claim 12 wherein the compound is used in targeted radionuclide therapy, wherein a patient is treated with the compound comprises a diagnostic radionuclide to identify the presence of a cancer or disease, followed by treatment with a compound comprising a therapeutic radionuclide to treat said cancer or disease.

15. The method of claim 14, wherein a patient, after confirming the presence of the cancer target using a compound comprising a therapeutic radionuclide, is treated with a compound comprising a therapeutic radionuclide to treat the said cancer or disease.

16. The method of claim 14, wherein the radionuclide is an isotope of I or Br.

17. The method of claim 16, wherein the therapy is achieved by injecting a patient with a compound radiolabeled with diagnostic radionuclide ¹²³I to identify a tumor or disease using SPECT imaging or using PET imaging if the radiolabel is ¹²⁴I, and then exchanging the radionuclide with a therapeutic radionuclide ¹²⁵I or ¹³¹I and injecting the said patient for therapy.

18. The method of claim 13, wherein the cancer type is cervical or esophageal cancer.

19. The method of claim 13, wherein the cancer type is glioblastoma.

20. A method of diagnosing or treating cancer, inflammatory diseases or Alzheimer's disease, wherein a patient is treated with a compound of claim 1.

21. A method of diagnosing or treating cancer, inflammatory disease or Alzheimer's disease, wherein a patient is treated with a compound of claim 12 comprising a diagnostic radionuclide to identify the presence of a cancer or disease, followed by treatment with a compound comprising a therapeutic radionuclide to treat the said cancer or disease.

22. The method claimed in claim 21, wherein the radionuclide is an isotope of I or Br.

23. The method claimed in claim 22, wherein the patient is injected with the compound of claim 12 radiolabeled with diagnostic radionuclide ¹²³I and the said tumor or disease is identified using SPECT imaging, or using PET imaging if the radiolabel is ¹²⁴I, and then exchanging the radionuclide with the compound radiolabeled with a therapeutic radionuclide ¹²⁵I or ¹³¹I and injecting the said patient for therapy.

24. The method claimed in claim 20, wherein the cancer is cervical or esophageal cancer.

25. The method claimed in claim 20, wherein the cancer is glioblastoma.

26. A method of preparing a radiolabeled compound according to claim 8 by the S_{E}Ar radio-iodination of the aromatic ring of the said compound or a precursor with N-chlorosuccinimide in trifluoroacetic acid.
